(19) United States
(12) Patent Application Publication
(10) Pub. No.: US 2004/0176700 A1
(43) Pub. Date: Sep. 9, 2004

(54) Diagnostic temperature probe

(76) Inventor: David Stuart Potter, Cowes (GB)

Correspondence Address: JVS Inventions, 1308 Henry Balch Drive, Orlando, FL, 32810, US

(21) Appl. No.: 10/481,097

(22) PCT Filed: Jun. 5, 2002
(86) PCT No.: PCT/GB02/02572

(30) Foreign Application Priority Data
Jun. 22, 2001 (GB) 0115308.9
Apr. 8, 2002 (GB) 0207982.0

(51) Int. Cl. A61B 5/00

(52) U.S. Cl. 600/549; 600/474

(57) ABSTRACT

An instrument for medical diagnosis of a body comprises measuring means (18) for measuring temperature of, or thermal radiation from, a region of the body at which the measuring means is directed and producing a measurement, and a hand-holdable housing (10). The housing contains: region indicating means, such as an LCD (14), for indicating to a user a sequence of at least three such regions at which the instrument is to be directed and such measurements are to be made; storing means (32) for storing the measurements so made (or data derived therefrom); processing means (26) for processing the stored measurements (or derived data) to ascertain correlation between the measurements; and result indicating means, such as the same, or a further, LCD, for indicating a result of the correlation to the user.

### DESCRIPTION

This invention relates to instruments for medical diagnosis.

The invention was originally conceived for diagnosing conditions in the human leg such as deep venous thrombosis, but the invention is also applicable to the diagnosis of conditions in elsewhere in the human body and in the animal body.

Hitherto, the early reliable diagnosis of conditions such as deep venous thrombosis and ischaemia in the leg has been problematic. It is known that such conditions affect the blood flow rate in the leg, which in turn affects the temperature of the leg. In the case of a leg having a deep venous thrombosis, the normal venous return of blood to the heart is blocked or restricted, and the dermal capillaries are forced to carry more of the blood, thereby forcing the blood nearer to the surface of the leg so that the surface temperature is higher than normal. On the other hand, in the case of a leg having ischaemia, the normal arterial flow of blood from the heart is blocked or restricted, so that the surface temperature of the leg is lower than normal. Based on this, it is known to capture a thermographic image of both legs which is then interpreted by a clinician searching for any temperature asymmetry between the two legs which might be indicative of thrombosis or ischaemia in one of the legs. It will be appreciated that such thermographic equipment is expensive, that the clinician must be skilled, that it is unlikely this technique would be successful in detecting generally symmetrical (or bilateral) thromboses in the two legs, and that the technique is inapplicable in the case of one-legged patients.

The present invention, or preferred features of it, is concerned with enabling diagnosis of conditions such as those mentioned above:

cheaply,

quickly,

reasonably reliably,

without the need for a skilled operator,

non-invasively,

at least in some embodiments, without contact with the patient, and

at least in some embodiments, in patients whether they have one or two legs.

In accordance with a first aspect of the invention, there is provided an instrument for medical diagnosis of a body, the instrument comprising measuring means for measuring temperature of, or thermal radiation from, a region of the body at which the measuring means is directed and producing a measurement, and a hand-holdable housing, the housing containing: region indicating means (such as an LCD) for indicating to a user a sequence of at least three such regions at which the instrument is to be directed and such measurements are to be made; storing means for storing the measurements so made (or data derived therefrom); processing means for processing the stored measurements (or derived data) to ascertain correlation between the measurements; and result indicating means (such as the same, or a further, LCD) for indicating a result of the correlation to the user.

This aspect of the invention can be thought of as being based on the realisation that the temperature profile of a series of regions along the limb of a person who is not suffering from one of these complaints is, within limits, predictable and so a deviation from the normal profile is indicative of an abnormal condition. Alternatively stated, this aspect of the invention can be thought of as being based on the realisation that the temperature profile of a series of regions along the limb of a person who is suffering from a particular one of these complaints is, within limits, predictable and so a similarity to such a profile is indicative of such a condition. The instrument is easy to use in the sense that the measurements are stored by the instrument and do not need to be written down by the user, and that the stored measurements are processed by the instrument so that the user does not need to do any calculation.

It will be appreciated that, in the case where three or more measurements are to be taken at different regions of the body, it is important to the correlation process that the measurements are taken in the correct sequence. The region indicating means facilitates this.

In one embodiment, the region indicating means is arranged to display a picture of each region at which the measuring means is to be directed. In another embodiment, the region indicating means is arranged to display a verbal description of each region at which the measuring means is to be directed. In the case of diagnosis of a limb, the region indicating means is preferably operable to indicate at least two longitudinal positions along the limb (such as mid thigh and mid calf), and even more preferably at least three or four such longitudinal positions (such as mid thigh, lower thigh, upper calf and mid calf). In this case, the processing means is preferably operable to calculate a difference between the measurement taken at each longitudinal position and at least one measurement taken at a different longitudinal position. The region indicating means is preferably also operable to indicate at least two circumferential positions around the limb (such as inside and outside, or front and back), and even more preferably at least three or four such circumferential positions (such as front-inside, front-outside, rear-inside and rear-outside). In this case, the processing means is preferably operable to calculate a difference between the measurement taken at each longitudinal and circumferential position and at least one measurement taken at a different longitudinal position and similar circumferential position.

In a further embodiment, the region indicating means is arranged to display a respective symbol for each region at which the measuring means is to be directed, the instrument being in combination with a set of markers each bearing a respective one of the symbols, each marker being self-adhesive so that it can be affixed to the respective region of the body. This feature facilitates consistency in the regions at which measurements are taken as between one set of measurements which may be taken by one user and another set of measurements which may be taken by a different user.

Preferably, the storing means is operable to store, and the processing means is operable to process, at least four, and more preferably at least five, and more preferably at least six such measurements (or derived data) in order to perform the correlation.

In one embodiment, the processing means is operable to ascertain correlation between the measurements and respective expected values indicative of a normal condition of the body, in which case the indicating means might provide indications such as “normal” or “abnormal”. Alternatively, the processing means may be operable to ascertain correlation between the measurements and respective expected values indicative of a particular abnormal condition of the body, such as deep venous thrombosis, in which case the indicating means might provide indications such as “DVT” or “Not DVT”. In the latter case, the processing means may also be operable to ascertain correlation between the measurements and respective expected values indicative of at least one further different abnormal condition of the body, such as ischaemia, in which case the indicating means might provide indications such as “DVT”, “Ischaemia” or “Not DVT or Ischaemia”. These manners of operation of the processing means may be combined, in which case the indicating means might provide indications such as “Normal”, “DVT”, “Ischaemia” or “Unknown Abnormal”.

The processing means may be operable to use another such stored measurement (or derived data) as a reference datum, the intention being that this additional measurement is indicative of body core temperature and might be taken from the patient's forehead or upper arm. The healthy human body attempts to keep its core temperature at about 37° C., but the actual temperature varies (a) generally between men and women, (b) between different individuals, (c) in dependence upon the time of day, and (d) in women, in dependence upon the phase in their menstrual cycles. In a feverish person, the body core temperature can vary quite considerably from the normal. Nevertheless, the temperature of the forehead of a relaxed person in a room at 20° C. is generally always about 2½° C. below their body core temperature. In order to diagnose conditions such as deep venous thrombosis or the like in the leg, the temperature drop from core temperature (or the forehead temperature) to the measurement taken nearest the crotch, and then the successive temperature drops along the leg from one region to the next are more informative than the actual temperatures of the various regions along the leg. Accordingly, by taking into account an indication of body core temperature, the processing means is better able to ascertain whether the measurements are indicative of an abnormal condition.

The instrument may further include means for measuring ambient temperature; the processing means being operable to use the measured ambient temperature (or derived data) as a correction factor. For a healthy relaxed person at an ambient room temperature of 20° C., the temperature progressively decreases down their leg to a temperature typically of 27° C. at their toes, but the toe temperature can vary considerably in dependence upon the ambient temperature. In order to diagnose conditions such as deep venous thrombosis or the like in the leg, it is believed that some function of ambient temperature and each temperature along the leg (or each successive temperature drop along the leg from one region to the next) may be more informative than the temperatures (or temperature drops) per se. Accordingly, by taking into account ambient temperature, the processing means may be better able to ascertain whether the measurements are indicative of an abnormal condition.

The measuring means may be operable without physical contact of the measuring means with the body. The lack of contact reduces the risk of cross-infection between different patients, and renders the instrument suitable for use with patients who have open wounds, rashes or fresh surgical scars in the regions to be measured, or who suffer from hyperalgesia (extreme sensitivity to touch) of the skin. In this case, the instrument preferably further includes distance indicating means for indicating to the user an intended measuring distance between the measuring means and the region of the body to be measured. In the case where the measuring means has a divergent field of view, the indication of a predetermined distance to the user enables a consistent size of target area of the body to be viewed. Preferably, the distance indicating means comprises a means for projecting a pair of beams of visible light that intersect at the intended measuring distance from the measuring means. Alternatively, if physical contact with the patient is acceptable, a shroud may be provided around the field of view of the measuring means for contact with the patient. In this case, the shroud is preferably disposable and replaceable.

The measuring means is preferably operable to measure thermal radiation from (rather than directly to measure temperature of) the body. As is well known, there is a natural relationship between the temperature of a body and the rate of heat energy radiated at certain wavelengths. In the case of human body temperatures, the wavelengths of peak radiation are between about 8 and 12 μm in the infra-red band. It is believed that the permeability of the skin and subcutaneous layer to such radiation is such that the amount of radiation is dependent not merely on the body surface temperature but on the temperatures over a depth to about 15 mm beneath the surface of the body. Accordingly, by measuring thermal radiation, the instrument is, to some extent, able to see under the skin. In particular, the measuring means may include an infra-red radiation sensor. Such sensors are available relatively cheaply off-the-shelf and have a suitably fast response time, typically of 0.5 s. Preferably, the instrument further comprises means for indicating to the user a predetermined distance (which might typically be chosen to be 60 mm) between the measuring means and the region of the body to be measured.

The instrument preferably includes an element (such as a push-button or key) which is manually operable by the user, the storing means being operable to store the current measurement (or derived data) in response to operation of the element.

The storing means is preferably operable to store the measurements for previous correlation processes in addition to the measurements for the current correlation process, and the instrument preferably further includes means for uploading the measurements to a separate apparatus, such as a PC that can then be used to analyse the measurements and present a record of the progression of the condition.

The instrument preferably includes a means for producing a real-time clock signal, the storing means being operable to store, for each correlation process, the current clock signal at, or at about, the time of that correlation process. A more detailed and informative log of the measurements is then compiled.

The instrument preferably includes a means for entering an indication of the identity of the body, the storing means being operable to store that identity indication in relation to the stored measurements. The instrument can therefore be used for different patients without the need for uploading the data between measurements on the different patients.

The processing means may be operable to perform its correlation processing in response to a predetermined number of temperatures having been measured.

The housing might, for example, be about the size of a mobile telephone or television remote controller, and would therefore be convenient to use, to carry and to store. Preferably, the measuring means is also contained by the housing. Preferably, the housing also contains means for generating or storing electrical energy (such as a battery or photovoltaic converter). Accordingly, a completely self-contained instrument can be provided.

When various of the above features are combined, it will be apparent that the instrument is very easy to use. The user is told by the LCD which region is to be measured, places the instrument in position to measure that region, presses the push-button or key, and then repeats these steps until the result of the diagnosis is indicated.

In accordance with a second aspect of the present invention, there is provided a method of diagnosing a deep venous thrombosis or similar complaint in a limb, comprising measuring the temperature of, or thermal radiation from, a plurality of prescribed isolated regions on the surface of the limb, the regions being spaced apart along and around the limb, and performing a predetermined algorithm on the measurements.

A specific embodiment of the present invention will now be described, purely by way of example, with reference to the accompanying drawings, in which:

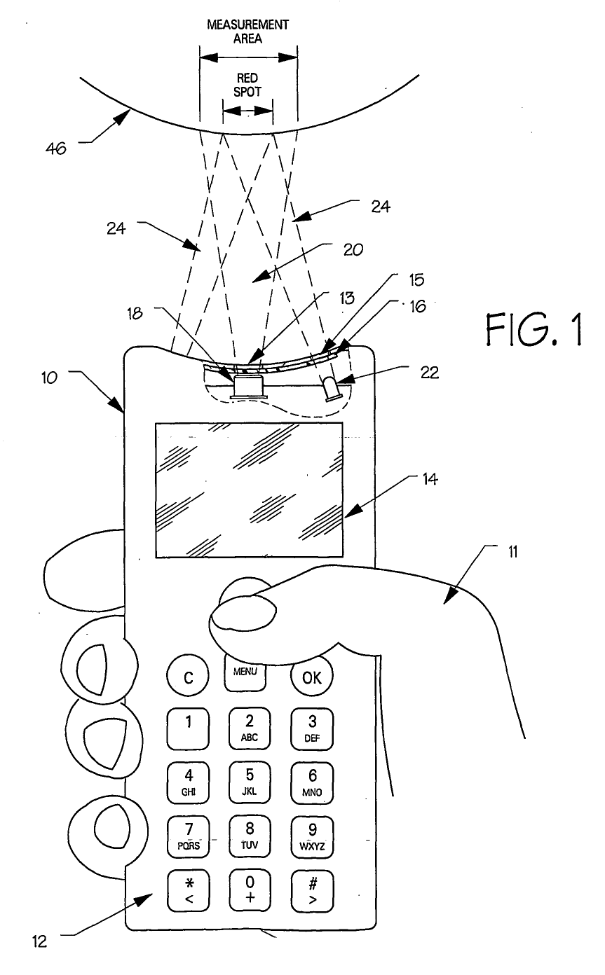

FIG. 1 is a top view of a medical diagnostic instrument, partly cut away, and showing part of a patient's leg and part of an operator's hand;

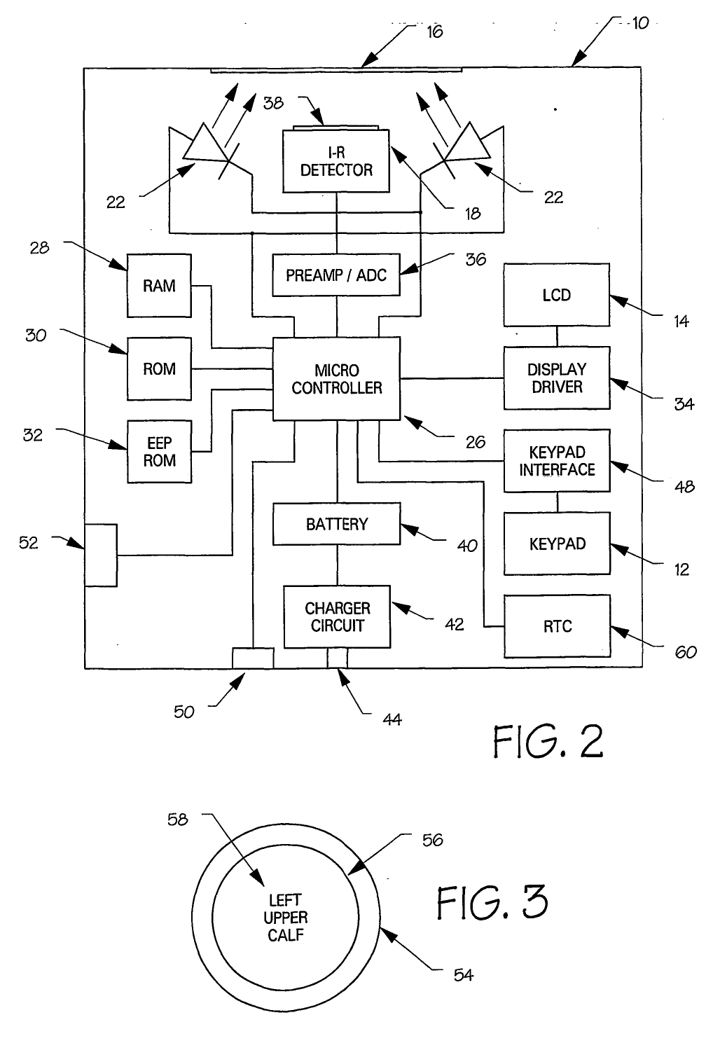

FIG. 2 is a schematic diagram of the functional elements of the instrument;

FIG. 3 is a plan view of a marker for use with the instrument;

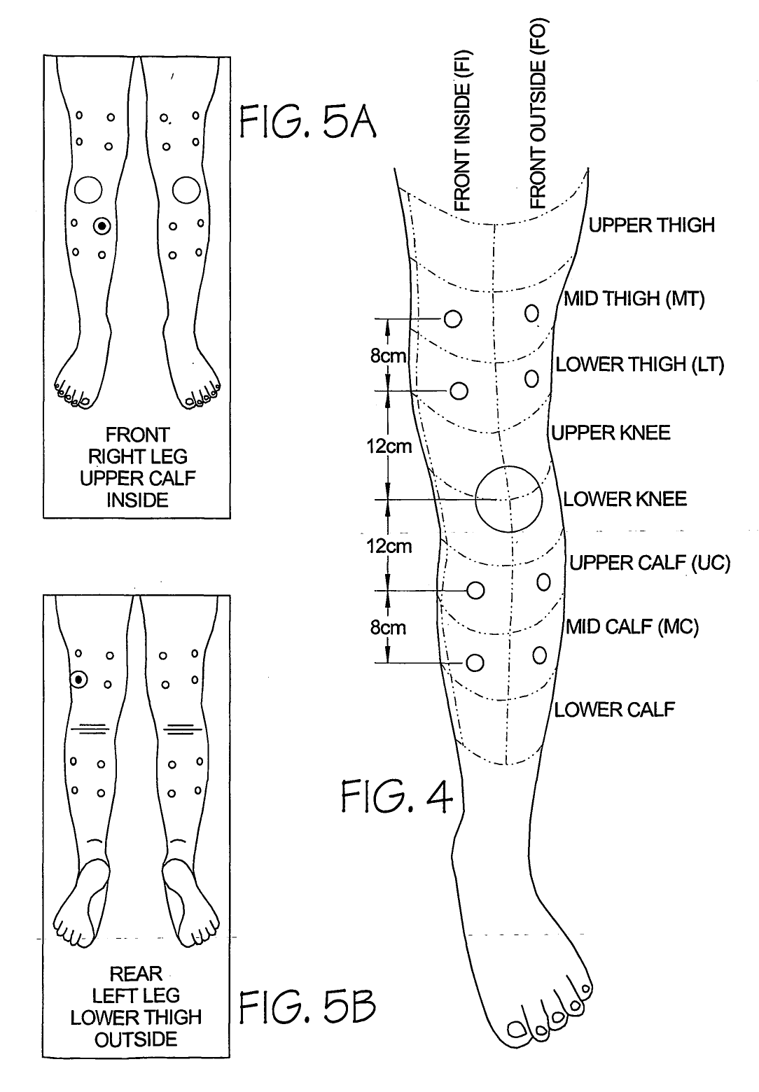

FIG. 4 is a front view of a person's left leg; and

FIGS. 5A & 5B are examples of displays that may be made by the instrument.

Referring to FIG. 1, the instrument has a housing 10 suitable for being held in the hand 11 of the operator similarly to a television remote controller. The top face of the housing 10 has a graphical liquid display (LCD) 14 and a keypad 12 of the mobile telephone type arranged so that the keys can be depressed by the user's thumb or forefinger. The front edge of the housing 10 has a central aperture 13, a pair of side apertures 15 and a protective window 16, which is permeable to infra-red radiation and red light, arranged behind the apertures. Inside the housing 10 and behind the central aperture 13, an infra-red detector 18 is positioned to detect incident infra-red radiation and has a conical field of view as indicated by the dashed lines 20. Also, inside the housing 10 and behind the side apertures 15, a pair of red LEDs 22 (one of which is shown in FIG. 1) are positioned so as to project individually-diverging, mutually-converging beams of light 24 which intersect at a suitable predetermined distance, such as 60 mm, from the front edge of the housing 10.

Referring now to FIG. 2, the housing 10 also contains a microcontroller 26 with associated ROM 30 storing the microcontroller's program, RAM 28 which is used as the microcontroller's working memory, and EEPROM 32 which is used to provide non-volatile memory for the measurements which are taken by the instrument. The microcontroller 26 is connected to the LCD 14 via a display driver 34. The infra-red detector 18 is connected to the microcontroller 26 via a preamplifier and analogue-to-digital converter circuit 36. The infra-red detector 18 may be implemented by a model 2M thermopile manufactured by Dexter Research Center of Dexter, Mich. 48130, USA, with a germanium filter 38 for effective transmission of radiation of wavelength 8 to 12 μm. The two LEDs 22 are connected in parallel or series to the microcontroller 26. The housing also contains a rechargeable battery 40, charger circuit 42 and power supply socket 44 which can be connected to a mains adapter to recharge the battery 40. The battery 40 is directly connected to the microcontroller 26 which controls the supply of power to the preamplifier and analogue-to-digital converter circuit 36, LEDs 22, RAM 28, ROM 30, EEPROM 32, display driver 34 and display 14. The keypad 12 is connected to the microcontroller 26 via an interface circuit 48.

The microcontroller 26 is preferably implemented as an ASIC and may include the RAM 28 and/or ROM 30 and/or EEPROM 32 and/or converter circuit 36 and/or display driver 34 and/or keypad interface circuit 48 in the same chip. Given the required functionality of these elements, as described below, the design of such an ASIC will be readily apparent to a person with normal skills in ASIC design.

Starting from a standby state when the instrument, the microcontroller 26 is configured and programmed by the ROM 30 to operate as follows:

A. In response to the “OK” key of the keypad 12 being pressed by the user, the microcontroller:

a. supplies power to the converter circuit 36, RAM 28, ROM 30, display driver 34 and display 14; and

b. causes the LCD 14 to display the message SEQUENCE MODE for a predetermined time while the circuitry settles down.

B. The microcontroller then causes the LCD 14 to display the message FOREHERD. The user is then expected:

a. to point the front edge of the instrument squarely at the patient's forehead;

b. to depress the “OK” key, whereupon the microcontroller causes the LEDs to illuminate;

c. to adjust the spacing between the instrument and the patient's forehead so that the beams 24 intersect to form a single dot on the patient's forehead; and

d. to release the “OK” key.

C. In response to release of the “OK” key, the microcontroller 26:

a. switches off the LEDs 22;

b. reads the output (M₀) from the converter circuit 36;

c. stores the value M₀ in the RAM 28; and

d. causes the LCD 14 to display the message UPPER THIGH.

The user is then expected:

e. to point the front edge of the instrument squarely at the rear of the patient's upper thigh 46;

f. to depress the “OK” key, whereupon the microcontroller causes the LEDs to illuminate;

g. to adjust the spacing between the instrument and the patient's upper thigh so that the beams 24 intersect to form a single dot on the patient's upper thigh; and

h. to release the “OK” key.

D. In response to release of the “OK” key, the microcontroller 26:

a. switches off the LEDs 22;

b. reads the output (Mi) from the converter circuit 36;

c. stores the value M₁ in the RAM 28; and

d. causes the LCD 14 to display the message LOSER THIGH.

The user is then expected:

e. to point the front edge of the instrument squarely at the rear of the patient's lower thigh;

f. to depress the “OK” key, whereupon the microcontroller causes the LEDs to illuminate;

g. to adjust the spacing between the instrument and the patient's lower thigh so that the beams 24 intersect to form a single dot on the patient's lower thigh; and

h. to release the “OK” key.

E. Step “D” is then repeated thrice, but to obtain stored values M₂, M₃ and M₄ for the lower thigh, behind the knee and the upper calf and with subsequent displays of BEHIND KNEE, UPPER CALF and LOSER CALF.

F. In response to the next release of the “OK” key, the microcontroller 26:

a. switches off the LEDs 22;

b. reads the output M (for the lower calf) from the converter circuit 36; and

c. stores the value Ms in the RAM 28.

G. The microcontroller 26 then:

a. performs a correlation process on the stored values, as will be described in more detail below;

b. then displays the result of the correlation process, such as:

NORMAL

SUSPECT D V T, or

SUSPECT ISCHAEMIA,

SUSPECT UNKNOWN; and

c. copies the values M₀ to M₅ from the RAM 28 to the EEPROM 32.

H. The microcontroller 26 then returns the instrument to the standby state after a predetermined period, such as one minute, (ready for a return to step “A” above) unless in the meantime the “OK” key is depressed, in which case a return is made to step “B” above.

For a healthy, resting adult having a body core temperature of 37° C. and with an ambient temperature of 20° C., typically the expected forehead temperature (E₀) would be 34½° C., the expected temperature of the toes would be 27° C., and the expected temperatures of the upper thigh (E₁), lower thigh (E₂), popliteal (E₃), upper calf (E₄) and lower calf (E₅) would progressively decrease from a value below 34½° C. to a value above 27° C. The temperature drop from the upper thigh to the lower calf, E₁-E₅, would typically be expected to be 3° C. In the case of a leg having a deep venous thrombosis, the normal venous return of blood to the heart is blocked or restricted, and the capillaries in the dermis region dilate and carry more blood, thereby causing the skin temperature to be higher than normal. On the other hand, in the case of a leg having ischaemia, the normal arterial flow of blood from the heart is blocked or restricted, so that the surface temperature of the leg is lower than normal. It is apparent that, by programming the microcontroller to process the measured values M1 to M1 in a manner which is dependent on the expected temperatures E₁ to E₅, it is possible to determine whether the measured values M₁ to M5 are indicative of deep venous thrombosis/ruptured Baker's cyst, of ischaemia, of some other abnormal condition, or of a normal condition. It is furthermore apparent that if the forehead measurement M₀ (or some other measurement indicative of body core temperature) and the normal forehead temperature E₀ are take into account in the processing, such conditions are likely to be more reliably detectable. It is also apparent that a significant factor in such diagnosis will be the differences between the successive measurements, i.e. M₁-M₂, M₂ -M₃, M₃ -M₄, M₄-M₅, and optionally M₀-M₁, and so the processing by the microcontroller should place significant weight on these differences, rather than merely the actual values of the measurements. It is moreover apparent that the particular site of any thrombosis or ischaemia along the leg will have some effect on at least some of the measurements M₁ to M₅, and accordingly it is anticipated that with suitable processing by the microcontroller 26 it may be possible to provide some indication of the site of the abnormal condition. Clinical trials should be conducted to obtain sets of measurements M₀ to M₅ for individuals known to be suffering from the different conditions and also for normal individuals. With such data, suitable algorithms performable by the microcontroller 26 for detecting the abnormal conditions will be apparent to a mathematician with normal skills in the art of data analysis and correlation techniques.

The infra-red detector 18 receives infra-red radiation from the part of the body being measured, and its sensitive element quickly assumes the temperature of the part of the body being measured. The detector 18 provides an output voltage V to the converter circuit 36 which is linearly related to the measured temperature M, i.e. V=aM+b, where a is a constant and b is a constant for a particular detector in a particular environment. As discussed above, temperature differences rather than actual temperatures are of primary significance. It will be appreciated that the difference in output voltages, say V₁-V₂, for a temperature drop M₁-M₂ is given by V₁-V₂=a(M₁-M₂), i.e. it is a proportional relationship. Given the constant of proportionality a, it is therefore possible for the microcontroller 26 to process the measurements as temperature drops in any desired units of measure, e.g. degrees Celsius, and if desired to cause the display 14 to indicate temperature drops in those units.

It will be appreciated that the decrease in temperature from the upper thigh to the toes is due to ambient cooling and is dependent on the ambient temperature. Again, in the case where the instrument is to be used at a known, generally constant, ambient temperature of say 20° C., as would generally be the case in hospital wards, the expected temperature differences along the legs in a healthy patient will be generally consistent. However, if the ambient temperature is lower or higher than this (as might be the case for general home use), the temperature differences along the leg will be lower or higher than those expected at an ambient temperature of 20° C. In order to correct for this, a sensor 50 for measuring ambient temperature may be provided in the housing 10 and connected to the microcontroller 26, in which case the microcontroller 26 is programmed to read the ambient temperature and to scale the measurements M₀ to M₅ in dependence upon the sensed ambient temperature. The sensor 50 is mounted so that it is thermally insulated from the housing 10 and is sited so that it is not significantly affected by the temperature of the hand of the user.

As mentioned above, in step “G” the microcontroller 10 copies the set of values M₀ to M₅ from the RAM 28 to the EEPROM 32, and so a series of sets of values can be built up in the EEPROM 32. The instrument has a data port 52 connected to the microcontroller 26 and which can be connected to a separate apparatus, such as a PC. The microcontroller 26 is programmed so that, in response to a command from the PC, it transmits the data from the EEPROM 32 to the PC, whereupon an application running on the PC can perform further analysis of the data.

The instrument may be provided with a real-time clock circuit 60 connected to the microcontroller 26. In this case, the microcontroller 26 may be programmed to read from the clock circuit 60 the date and time of each measurement that is taken and to store the time and date in the EEPROM 32 along with the measurement. The time/date data may then also be uploaded via the data port 52. The time and date of the clock circuit 60 may be set via the keypad 12, for example using the “menu” key and the numeric keys of the keypad 12.

The instrument may also be configured for use with different patients. For example, the name (or other way of identifying) a patient may be stored in the EEPROM 32 in the instrument, for example using the “menu” key and the alphabetic keys of the keypad 12. Then, each time a set of measurements is to be taken, the user is prompted to select the name of the patient from a list of the patients' names stored in the EEPROM 32 and displayed on the LCD 14, for example using the arrow keys and the “OK” key of the keypad 12. The selected patient's name is then stored in the EEPROM 32 along with the measurements and may also be uploaded via the data port 52.

In addition to operating in the mode described above, the instrument is also arranged to operate in a second, temperature-differential mode. In order to switch to the temperature-differential mode, the user depresses the “OK” key a second time during step “A” above, whereupon:

I. The microcontroller causes the LCD 14 to display the message TEMP DIFF MODE for a predetermined time while the circuitry settles down.

J. The microcontroller then causes the LCD 14 to display the message FIRST READING. The user is then expected:

a. to point the front edge of the instrument squarely at one region to one side of the patient's body;

b. to depress the “OK” key, whereupon the microcontroller causes the LEDs to illuminate;

C. to adjust the spacing between the instrument and the region of the body so that the beams 24 intersect to form a single dot on the patient's body; and

d. to release the “OK” key.

K. In response to release of the “OK” key, the microcontroller 26:

a. switches off the LEDs 22;

b. reads the output (M_{L}) from the converter circuit 36;

c. stores the value M_{L} in the RAM 28; and

d. causes the LCD 14 to display the message SECOND READING.

The user is then expected:

e. to point the front edge of the instrument squarely at the region to the other side of the patient's body which is symmetrical to the first region;

f. to depress the “OK” key, whereupon the microcontroller causes the LEDs to illuminate;

g. to adjust the spacing between the instrument and the region of the body so that the beams 24 intersect to form a single dot on the patient's body; and

h. to release the “OK” key.

L. In response to release of the “OK” key, the microcontroller 26:

a. switches off the LEDs 22;

b. reads the output (M_{R}) from the converter circuit 36; and

c. stores the value M_{R} in the RAM 28.

M. The microcontroller 26 then:

a. compares the stored values M_{L} and M_{R} and calculates a temperature difference T of how much higher the temperature indicated by the value M_{L} is than the temperature indicated by the value M_{R}; and

b. then displays the result of the comparison, such as:

FIRST HIGHER BY T C, if T>0;

SECOND HIGHER BY T C, if T<0; or

EQUAL, if T=0 within limits.

N. The microcontroller 26 then returns the instrument to the standby state after a predetermined period, such as one minute, (ready for a return to step “A” above) unless in the meantime the “OK” key is depressed, in which case a return is made to step “J” above.

The temperature differential mode of operation of the instrument may be used for comparing temperatures at symmetrical regions on the two legs of the patient so as to attempt to diagnose complaints such as deep venous thrombosis/ischaemia by asymmetry. This mode may also be used to diagnose other complaints such a malignant tumour in a breast which causes a higher temperature than the temperature of the surrounding tissue of the breast or of the symmetrical location on the other breast. Furthermore, in say the treatment of toe ulcers in a diabetic, this mode may be used periodically to measure the temperature difference between the patient's forehead (or upper arm) and their toes so as to monitor the patient's response to the treatment.

It will be appreciated that many modifications and developments may be made to the embodiment of the invention described above.

For example, in order to deal with the case where the user thinks that one of the measurements may have been inappropriately taken, the microcontroller 26 may be programmed to be responsive to depression of the “C” (or Cancel) key of the keypad 12 to cancel the latest measurement and allow it to be taken again. It may also be programmed to be responsive to an even longer depression of the push-button 12 to cancel all of the measurements taken so far and to revert to step “B” or “J” as appropriate.

Also, rather than relying mainly on the use of the “OK” key, the other keys of the keypad 12 may be employed for various functions.

Furthermore, although the use of an infra-red radiation detector is preferred for the reasons explained above, other types of temperature measuring device may be used.

Moreover, although the use of an LCD is preferred, other means of indicating messages and/or results to the user may be employed.

Additionally, although the taking of five measurements along the patients leg has been described above, the instrument may be programmed for the taking of other numbers of measurements, as will be described in more detail below.

Furthermore, the radiation detector may be fitted with a lens and/or a reflective funnel to focus the view of the radiated energy from the target and prevent the detector seeing any part of the housing other than the permeable window 16.

Also, instead of or in addition to prompting the user with a description of the region at which a measurement is to be taken, such as UPPER THIGH, the microcontroller 26 may be programmed, as will be described in more detail below, to cause the LCD 14 to display a picture of, for example, the legs of a person, and to cause a marker to flash on the picture, before each measurement is taken, showing the region at which the measurement is to be taken. Alternatively, the instrument may be supplied in combination with a set of self-adhesive markers 54 as shown in FIG. 3, for example of sticking plaster with a high transmissibility to infra-red radiation. Each marker 54 is printed with a respective symbol or symbols 58, such as the numbers 1 to 11, or the names “forehead”, “left upper thigh”, “left lower thigh”, “left behind knee”, “left upper calf”, “left lower calf”, “right upper thigh”, “right lower thigh”, “right behind knee”, “right upper calf” and “right lower calf”. A set of instructions may also provided explaining where the markers 54 should be affixed to the patient's body. In the case, the microcontroller 26 is programmed to prompt the user using the symbol, symbols or names as printed on the markers 54. The markers 54 may also be printed with a circle 56 to assist in aligning the spot produced by the LEDs 22.

A preferred method of operation and use of the instrument will now be described in more detail with reference to FIGS. 4 to 5B. FIG. 4 is a front view of a person's left leg which can be considered as being divided lengthwise into a number of slices that are labelled upper thigh, mid thigh, lower thigh, upper knee, lower knee, upper calf, mid calf and lower calf. The leg can also be considered as being divided circumferentially into four quadrants, two of which are shown, labelled front inside (FI) and front outside (FO), and the other two of which are the rear inside (RI) and the rear outside (RO). These eight slices and four quadrants together divide the surface of the leg into thirty-two zones, and measurements are taken generally at the centres of some of the zones. Trials have been carried, and it has been found out that an effective diagnosis can be made by taking sixteen measurements at the zones in the mid thigh (MT), lower thigh (LT), upper calf (UC) and mid calf (MC) slices, omitting the zones in the upper thigh, upper knee, lower knee and lower calf slices. The positions at which measurements should be taken on the front of the leg are shown by small circles in FIG. 4. For an average-sized person, the measurement positions on the lower thigh and upper calf are about 12 cm above and below the knee; the measurement positions on the mid thigh are about 8 cm above the lower thigh measurement positions; and the measurement positions on the mid calf are about 8 cm below the upper calf measurement positions. More generally, the mid thigh measurement positions are about half way between the knee and the crotch; the lower thigh measurement positions are about 30% of the way from the knee to the crotch; the upper calf measurement positions are about 30% of the way from the knee to the upper foot; and the mid calf measurement positions are about half way between the knee and the upper foot.

The measurements are taken using the instrument described with reference to FIGS. 1 and 2. Before taking the measurements, the patient lies down on a bed (away from draughts and any sources of direct heat) with both legs bared and with their heels supported by a cushion to allow air to circulate around the legs for ten minutes. The measurements on the fronts of the legs are then taken in an order that is prompted by the instrument. The patient is then turned over, and the measurements on the backs of the legs are taken, again in an order that is prompted by the instrument.

The following table sets out the temperatures that were recorded for a patient who was subsequently diagnosed using a conventional method as having a DVT in their left leg:

| | | Left Leg | | | | Right Leg | | | |
|---|---|---|---|---|---|---|---|---|---|
| | | RO | FO | FI | RI | RI | FI | FO | RO |
| Slice | MT | 34.4° C. | 31.3° C. | 32.3° C. | 33.2° C. | 34.5° C. | 31.8° C. | 32.0° C. | 31.8° C. |
| | LT | 32.5° C. | 29.9° C. | 31.6° C. | 32.2° C. | 33.7° C. | 31.0° C. | 30.9° C. | 31.1° C. |
| | UC | 33.7° C. | 32.7° C. | 33.7° C. | 33.2° C. | 31.2° C. | 30.1° C. | 31.1° C. | 30.4° C. |
| | MC | 34.2° C. | 33.2° C. | 32.8° C. | 33.2° C. | 31.2° C. | 29.3° C. | 30.0° C. | 30.5° C. |

On the basis of these results and those of thirty-two other patients, all of whom were also diagnosed using conventional methods, the following algorithm was developed: a patient is diagnosed as not having a DVT in a particular leg if, in each quadrant of that leg, MT-LT ≧T1 and MT-UC≧T2 and MT-MC≧T3, where T1, T2 and T3 are constants. On the basis of the tests conducted so far, the values chosen for T1, T2 and T3 are −0.4° C., 0.1° C. and 0.6° C., respectively.

In respect of the patient mentioned above, this algorithm produces the following results:

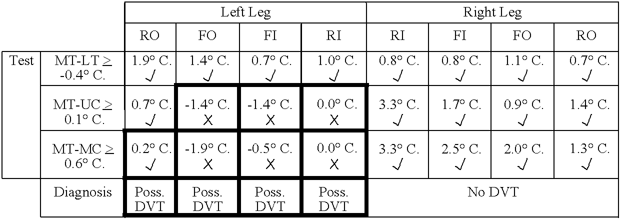

In respect of all thirty-three patients, the algorithm was applied to their measurements and they were also diagnosed using conventional methods, and the results are summarised in the following table:

| | | Conventional Diagnosis | |
|---|---|---|---|
| | | DVT | No DVT |
| The | Possible | 33% | 27% |
| Current | DVT | (Algorithm correct) | (Algorithm false - |
| Algorithm | | | non-critical) |
| | No | 0% | 39% |
| | DVT | (Algorithm false - | (Algorithm correct) |
| | | critical) | |

Of overriding importance is that fact that none of the patients was diagnosed by the current algorithm as not having a DVT when in fact they did. However, also of great significance is that fact that nearly 40% of patients were correctly diagnosed as not suffering from a DVT using the simple, quick and inexpensive test provided by the embodiment of the invention, obviating the need for more complicated, skilled and expensive testing. Of the remaining 60% of patients who were diagnosed by the current algorithm as possibly suffering from a DVT, over one half were subsequently found indeed to be so suffering.

In order to facilitate the taking of the measurements, the instrument of FIGS. 1 and 2 is programmed to prompt the user, using the display 14, as to the position where each measurement is to be taken. Such prompting may be verbal, such as:

Front—Right leg—Mid thigh—Outside

before the first measurement is taken, then:

Front—Right leg—Lower thigh—Outside

after the first measurement is taken but before the second measurement is taken, then:

Front—Right leg—Upper calf—Outside

after the second measurement is taken but before the third measurement is taken, then:

Front—Right leg—Mid calf—Outside

after the third measurement is taken but before the fourth measurement is taken, then:

Front—Right leg—Mid thigh—Inside

after the fourth measurement is taken but before the fifth measurement is taken, and so on.

The instrument is programmed to perform the algorithm on the measurements that are taken and then to display the results, such as:

Right leg: No DVT

Left leg: Possible DVT

The instrument preferably prompts the user to take the four measurements along each longitudinal line in sequence, because it is possible that a suspected DVT may be diagnosed in a leg after one, two or three longitudinal lines of measurements have been taken without the need to complete the other measurements. The instrument can then perform the algorithm immediately after each set of four measurements on the same longitudinal line has been taken. For example, in the case of the patient mentioned above, a suspected DVT in the left leg could have been diagnosed after only four measurements in any of the four quadrants without the need to take the other twelve measurements. Also, usually a patient would be complaining of pain or swelling in only one leg, in which case measurements would be take only on that leg. The prompt as to the leg on which the measurements are to be taken may therefore be omitted.

Instead of, or in addition to, displaying verbal prompts, the instrument may be programmed to cause the display 14 to show a picture to assist in taking the measurements at the correct locations. FIGS. 5A and 5B shows examples of pictorial and verbal prompts for two of the measurements to be taken. The appropriate position is highlighted, for example by emboldening, encircling, flashing and/or a change of colour.

The algorithm described above uses, for each leg, measurements taken in four quadrants and in four particular longitudinal positions along the leg. It is believed that other algorithms may be developed using more or less circumferential divisions around the leg and more or less or different longitudinal positions along the leg. Of course, the fewer measurements that are taken, the less reliable the algorithm can be expected to be, and the more measurements that are taken, the more time consuming the measuring process will be. It is also believed that other algorithms may be developed for diagnosing other conditions that affect the blood flow in the leg or in other parts of the body.

In a further modification of the instrument described about, the LEDs 22 and intersecting light beams 24 are not employed. Instead, the housing 10 is provided with a frusto-conical shroud having an internal reflective surface that surrounds the field of view 20 of the detector 18. In use, the distal rim of the shroud is placed in contact with the patient's skin around the spot where a measurement is taken. The shroud may be a clip fit to the housing 10 and be disposable so that a fresh shield may be used for each patient.

The foremost use of the instrument described above in likely to be in primary care for screening out suspect DVT patients who might otherwise have been referred to hospital for expensive scans. Other medical uses for the instrument may justify a model where the set reference programme can easily be changed from one application to another merely by inserting a different memory card, physically similar perhaps to a SIM card used mobile telephones. Other uses include the after-care monitoring in hospital and in home over several weeks of post-operative patients who are prone to developing DVTs after undergoing hip replacements or knee surgery, monitoring the progression/regression of toe ulcers on diabetics, and checking breast tumours for being benign or cancerous; cancerous tumours attract a greater blood supply than in surrounding tissue and therefore show up as warmer areas than benign growths. In each instance the instrument would prompt the user where and in what sequence to take a number of readings, and display automatically whether or not symptoms of that particular medical complaint are present without any interpretation of the measurements by the user. Other options include downloading the readings to a printer or to a computer for long-term storage or further analysis by physician or skilled clinician.

It should be noted that the embodiment of the invention has been described above purely by way of example and that many other modifications and developments may be made thereto within the scope of the present invention.

1. An instrument for medical diagnosis of a body, the instrument comprising measuring means (18) for measuring temperature of, or thermal radiation from, a region of the body at which the measuring means is directed and producing a measurement, and a hand-holdable housing (10), the housing containing:
region indicating means (14) for indicating to a user a sequence of at least three such regions at which the instrument is to be directed and such measurements are to be made;
storing means (32) for storing the measurements so made (or data derived therefrom);
processing means (26) for processing the stored measurements (or derived data) to ascertain correlation between the measurements; and
result indicating means (14) for indicating a result of the correlation to the user.

2. An instrument as claimed in claim 1, wherein the region indicating means is arranged to display a picture (FIGS. 5A,5B) of each region at which the measuring means is to be directed.

3. An instrument as claimed in claim 1 or 2, wherein the region indicating means is arranged to display a verbal description (FIGS. 5A,5B) of each region at which the measuring means is to be directed.

4. An instrument as claimed in claim 2 or 3, wherein, in the case of diagnosis of a limb, the region indicating means is operable to indicate at least two longitudinal positions along the limb.

5. An instrument as claimed in claim 4, wherein the processing means is operable to calculate a difference between the measurement taken at each longitudinal position and at least one measurement taken at a different longitudinal position.

6. An instrument as claimed in any of claim 4 or 5, wherein the region indicating means is operable to indicate at least two circumferential positions around the limb.

7. An instrument as claimed in claim 6, wherein the processing means is operable to calculate a difference between the measurement taken at each longitudinal and circumferential position and at least one measurement taken at a different longitudinal position and similar circumferential position.

8. An instrument as claimed in any preceding claim, wherein the region indicating means is arranged to display a respective symbol for each region at which the measuring means is to be directed, the instrument being in combination with a set of markers each bearing a respective one of the symbols, each marker being self-adhesive so that it can be affixed to the respective region of the body.

9. An instrument as claimed in any preceding claim, wherein the storing means is operable to store, and the processing means is operable to process, at least four, and more preferably at least five, and more preferably at least six such measurements (or derived data) in order to perform the correlation.

10. An instrument as claimed in any preceding claim, wherein the processing means is operable to ascertain correlation between the measurements and respective expected values indicative of a normal condition of the body.

11. An instrument as claimed in any preceding claim, wherein the processing means is operable to ascertain correlation between the measurements and respective expected values indicative of a particular abnormal condition of the body.

12. An instrument as claimed in claim 11, wherein the processing means is operable to ascertain correlation between the measurements and respective expected values indicative of at least one further different abnormal condition of the body.

13. An instrument as claimed in any preceding claim, wherein the processing means is operable to use another such stored measurement (or derived data) as a reference datum.

14. An instrument as claimed in any preceding claim, further including means (50) for measuring ambient temperature; the processing means being operable to use the measured ambient temperature (or derived data) as a correction factor.

15. An instrument as claimed in any preceding claim, wherein the measuring means is operable without physical contact of the measuring means with the body.

16. An instrument as claimed in any preceding claim, further including an element (12) which is manually operable by the user; the storing means being operable to store the current measurement (or derived data) in response to operation of the element.

17. An instrument as claimed in any preceding claim, wherein the storing means is operable to store the measurements for previous correlation processes in addition to the measurements for the current correlation process, and further including means (52) for uploading the measurements to a separate apparatus.

18. An instrument as claimed in any preceding claim, wherein the instrument includes a means (60) for producing a real-time clock signal, and the storing means is operable to store, for each correlation process, the current clock signal at, or at about, the time of that correlation process.

19. An instrument as claimed in any preceding claim, wherein the instrument includes a means (12) for entering an indication of the identity of the body, and the storing means is operable to store that identity indication in relation to the stored measurements.

20. An instrument as claimed in any preceding claim, wherein the processing means is operable to perform its correlation processing in response to a predetermined number of temperatures having been measured.

21. A method of medical diagnosis of a body using a hand-holdable instrument that can measure temperature of, or thermal radiation from, a region of the body at which the instrument is directed and produce a measurement, the method comprising the steps of:
indicating to a user a sequence of at least three such regions at which the instrument is to be directed and such measurements are to be made;
storing the measurements so made (or data derived therefrom);
processing the stored measurements (or derived data) to ascertain correlation between the measurements; and
indicating a result of the correlation to the user.
